(19) United States
(12) Patent Application Publication
(10) Pub. No.: US 2022/0111097 A1
(43) Pub. Date: Apr. 14, 2022

(54) System and Method of Managing Requirements for Foodstuffs with Processing Line Changes

(71) Applicant: Jeffrey Hassell, New Lenox, IL (US)

(72) Inventor: Jeffrey Hassell, New Lenox, IL (US)

(21) Appl. No.: 17/560,049

(22) Filed: Dec. 22, 2021

Related U.S. Application Data
(63) Continuation-in-part of application No. 17/377,332, filed on Jul. 15, 2021.
(60) Provisional application No. 63/052,346, filed on Jul. 15, 2020.

(51) Int. Cl. A61L 2/24; A23L 33/00; H04L 67/306

(52) U.S. Cl. CPC A61L 2/24; A23L 33/40; A61L 2202/14; A23V 2002/00; H04L 67/306

(57) ABSTRACT

A system and a method of managing requirements for foodstuffs with processing line changes output assessment reports and work orders for an efficient processing of products. The system includes at least one computing system which manages and processes information inputted by the user. A user can create product profiles which include attribute information. The attribute information can be entered or selected by the user. The computing system compares the product profiles amongst each other in order to identify compatible product profiles and incompatible product profiles. The computing system then generates at least one processing instruction for each product profile based on the compatible product profiles and the incompatible product profiles. The processing instruction for each product profile is outputted as an assessment report and a work order which can be used to instruct employees on how to clean and verify products in a production line.

The current application is a continuation-in-part (CIP) application of a U.S. non-provisional application Ser. No. 17/377,332 filed on Jul. 15, 2021. The U.S. non-provisional application Ser. No. 17/377,332 claims a priority to the U.S. provisional patent application Ser. No. 63/052,346 filed on Jul. 15, 2020.

### FIELD OF THE INVENTION

The present invention generally relates to automation systems and business analysis methods. More specifically, the present invention provides a system and method of managing requirements for foodstuffs with processing line changeovers.

### BACKGROUND OF THE INVENTION

Over the last decade, undeclared allergens have quickly become the top reason for food recalls in the United States due to the potential danger of causing an allergic reaction to consumers. Researchers estimate that 32 million Americans have food allergies, requiring approximately 200,000 people to seek emergency medical care for allergic food reactions each year. Undeclared allergens can come from various sources, including cross contact from equipment that was not properly cleaned to remove allergenic material from a previous product. Dealing with allergen concerns can lead to high equipment requirements. Beyond allergens, equipment requirements may also be affected when changing from one product to another on a food processing line. Improper equipment set up can affect product quality, product safety, and line efficiency. Furthermore, various equipment such as blender type, filler type, filters, sifters, metal/x-ray detectors, and barcode readers have a significant effect on meeting product specifications.

In the past, food and dietary supplement manufacturing plants have relied on large, multi-paged matrices to determine product changeover requirements for production lines or production areas. The use and maintenance of these matrices is onerous and may lead to errors due to the manual process. Further, a standard matrix does not provide any further instruction or guidance on how to proceed based on the results found. The changeover process is typically a manual system which requires referencing various information sources to determine changeover requirements. This process also requires a person with a high level of understanding of regulations to accurately execute it properly, requiring companies to have additional staff which otherwise may not be required. The current food industry environment requires consideration of differences in allergens, product certifications, and food safety equipment which can all differ between products. It is a cumbersome process to accurately evaluate the information to make a proper decision on how to clean the production line, what level of verification is needed, and what equipment is needed for the next product. These are all critical factors when manufacturing safe food for consumers.

An objective of the present invention is to provide systems and methods to control the risk of allergen cross contact from food processing equipment. The present invention allows for proper identification of manufacturing equipment set up from product to product. Another element which is prevalent in the food industry is the need to control different product certifications from one product to the next. Some of these certifications are food-safety related, such as Gluten-Free or Sugar-Free. Other certifications are related to specific consumer segments such as Kosher, Halal, Non-Genetically Modified, or Organic. What all these certifications have in common is that material carryover from previous products on processing equipment can result in the product not meeting the full requirements of the certification.

The typical process for processing line changeovers includes scheduling a next product for manufacturing, evaluating what actions are needed to change from one product to another based on product attributes, determining what equipment may be needed to complete the job, cleaning and sanitizing the processing line to the prescribed level based on product attributes, and verifying that the processing line was cleaned to the prescribed level based on product attributes. Cleaning may range from no cleaning at all to wiping or brushing down equipment to a full disassembly cleaning with a commercial detergent and sanitizer. Verification may range from a visual inspection to swabbing equipment for allergen or microorganism cross contamination. Once the equipment is verified to be appropriately cleaned, it may be set up and used for production of the next product.

The present invention allows for the effective management of a wide variety of allergens and certifications throughout a company's product portfolio on the same production line, minimizing the potential for allergen cross-contact and certification non-compliance.

### BRIEF DESCRIPTION OF THE DRAWINGS

FIG. 1 is a system diagram of the present invention.

FIG. 2 is a flowchart illustrating the overall process for the method of the present invention.

FIG. 3 is a flowchart illustrating the subprocess of a user selecting attribute information for the product profiles.

FIG. 4 is a flowchart illustrating the subprocess of a user entering attribute information for the product profiles.

FIG. 5 is a flowchart illustrating the subprocess for outputting a changeover assessment.

FIG. 6 is a flowchart illustrating the subprocess for outputting a simulated production-line schedule.

FIG. 7 is an exemplary illustration of product profiles.

FIG. 8 is an exemplary illustration of cleaning and verification attribute information.

FIG. 9 is an exemplary illustration of a changeover assessment.

FIG. 10 is an exemplary illustration of a simulated production-line schedule.

### DETAIL DESCRIPTIONS OF THE INVENTION

All illustrations of the drawings are for the purpose of describing selected versions of the present invention and are not intended to limit the scope of the present invention.

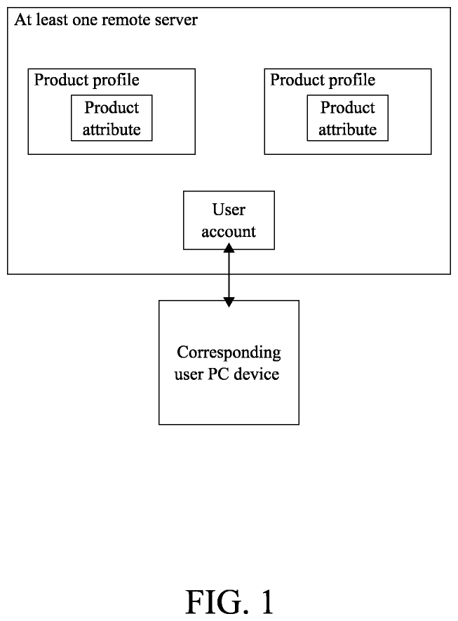

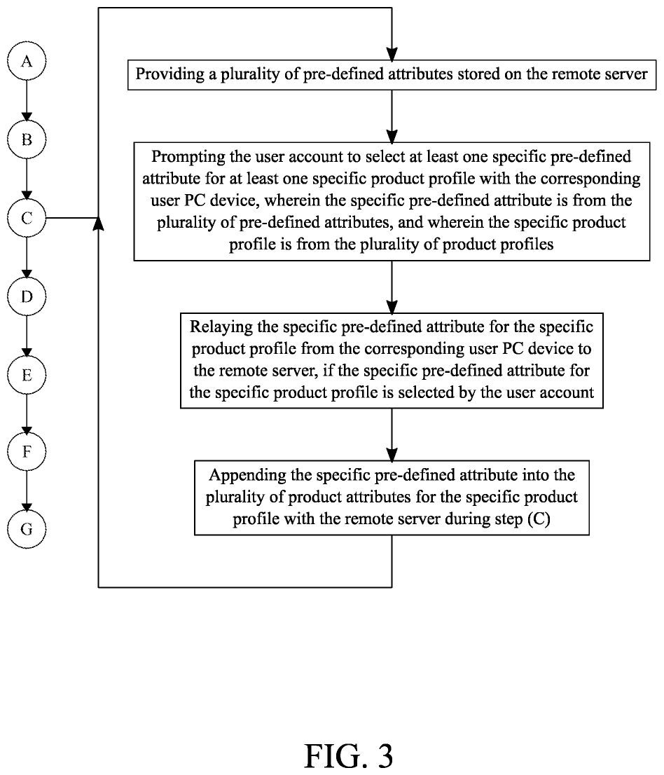

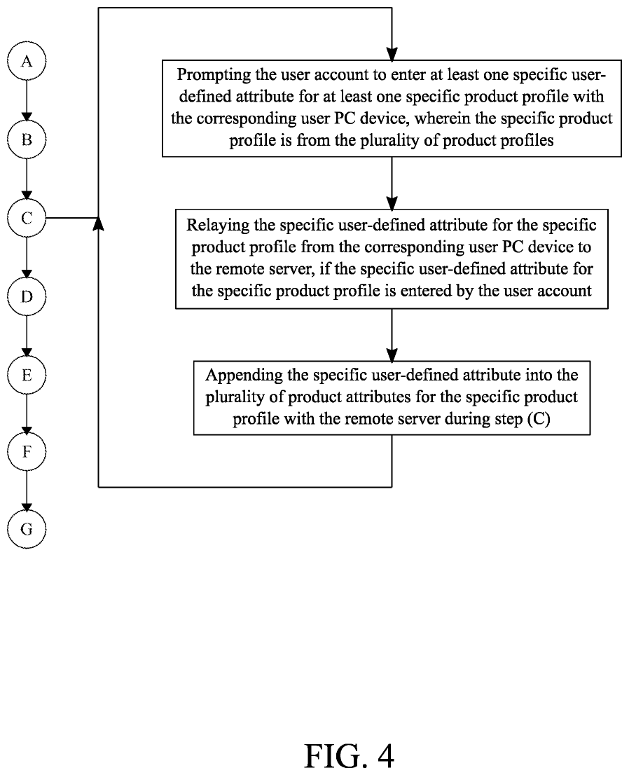

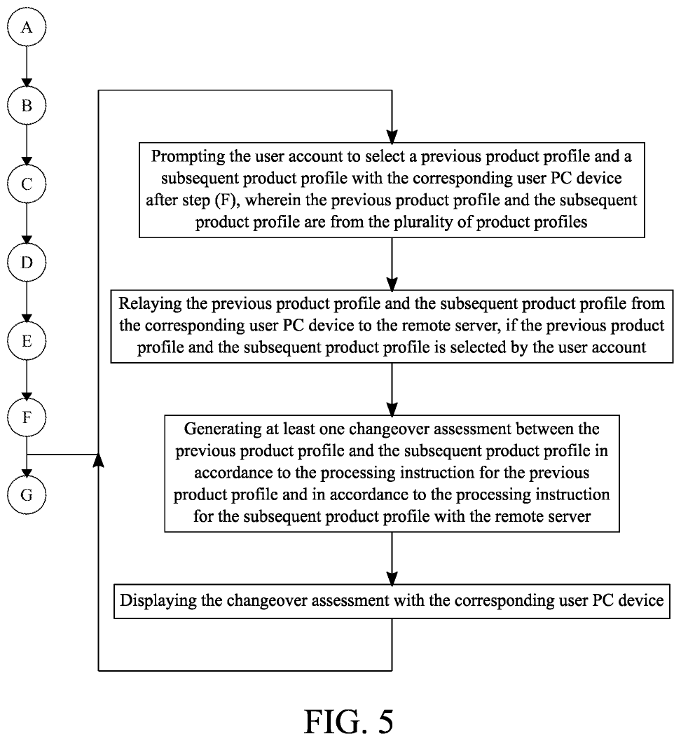

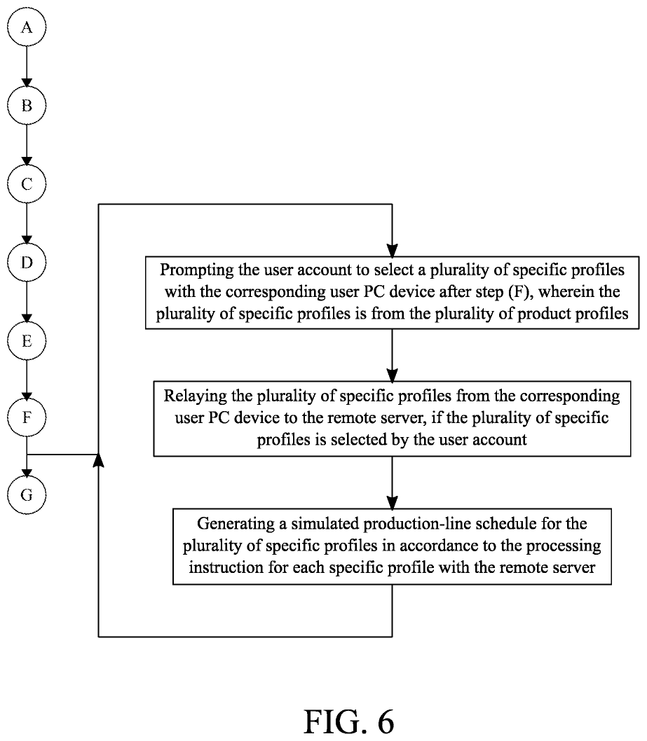

In reference to FIGS. 1 through 8, the present invention is a system and method of managing requirements for foodstuffs with processing line changes. With reference to FIG. 1, the system of the present invention includes at least one computing device which is preferably at least one remote server that manages at least one user account (Step A). The user account is associated with a corresponding user personal computing (PC) device. The corresponding user PC device may be any computing device such as, but not limited to, a desktop computer, a notebook computer, a smartphone, or a mobile tablet. The remote server is a cloud server that manages and processes data communicated from the corresponding user PC device. Alternatively, in a local embodiment of the present invention, the at least one computing device may be any computing device such as, but not limited to, a desktop computer, a notebook computer, a smartphone, or a mobile tablet rather than at least one remote server. In this case, the computing device must include sufficient hardware in order to execute the method of the present invention.

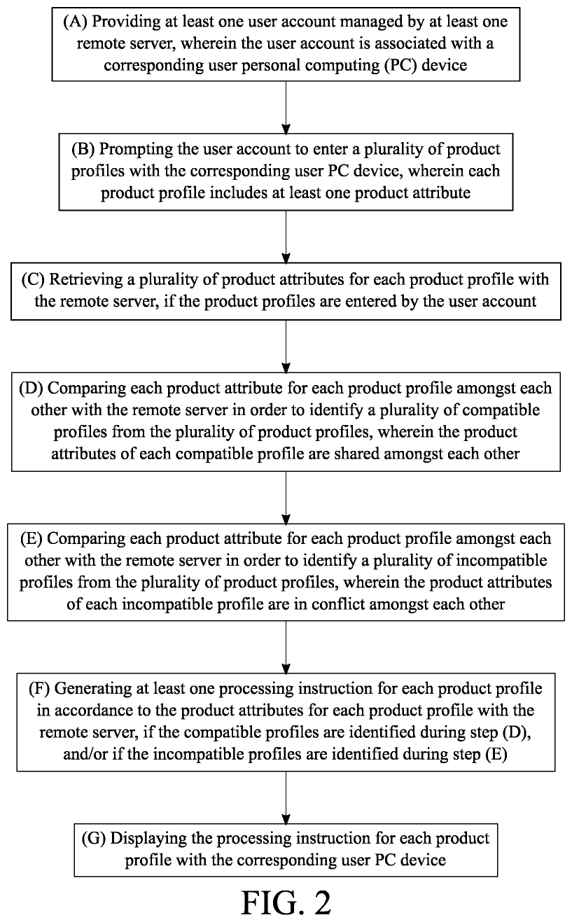

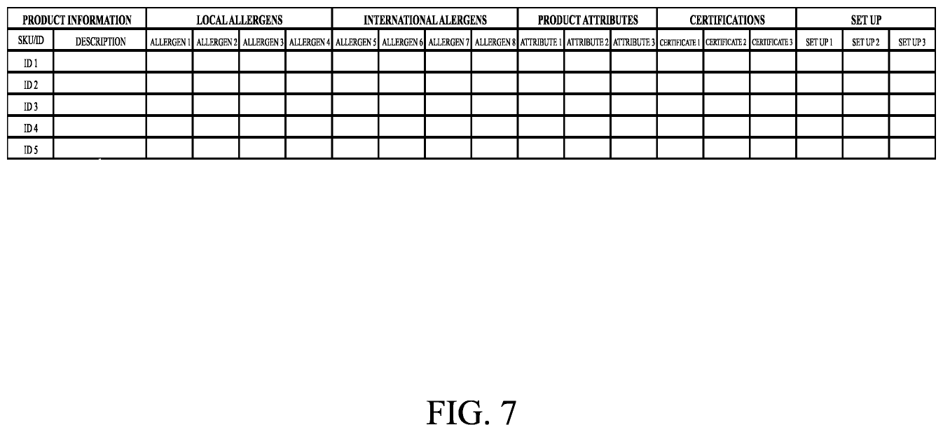

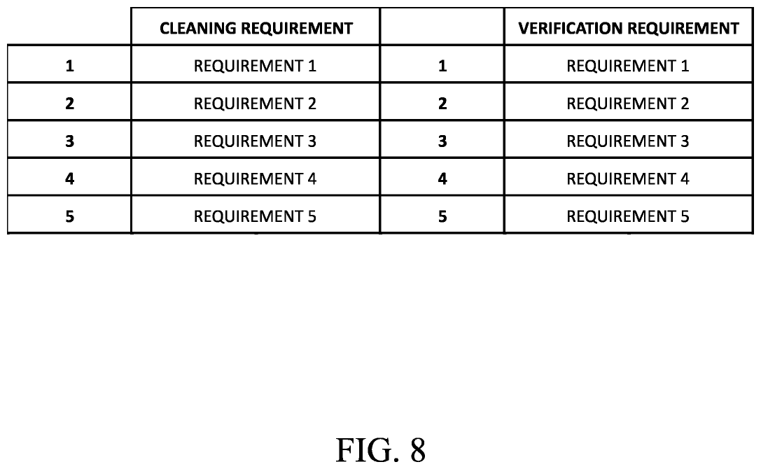

With reference to FIG. 2, the method of the present invention follows an overall process for managing requirements for foodstuffs with processing line changes. First, the corresponding user PC device prompts the user account to enter a plurality of product profiles (Step B). With reference to FIG. 7, the plurality of product profiles is a set of profiles that can be created with product information by the user account. For instance, each product profile includes at least one product attribute. The product attribute may be any information such as, but not limited to, allergens, sensitizers, product certifications, equipment requirements (including color coding policies), personnel requirements, or physical/chemical attributes of a product. The remote server retrieves a plurality of product attributes for each product profile, if the product profiles are entered by the user account (Step C). In more detail, the user, through the user account, can create product profiles for each product the user works with and while creating the product profile, the user can fill in or select the plurality of product attributes. Furthermore, after each product profile is created, the remote server generates a product ID for each product profile. With reference to FIG. 8, the plurality of product attributes is used to determine the level of sanitation and verification required and any other line changes needed to produce the next food product. After the user has created the product profiles, the remote server compares each product attribute for each product profile amongst each other in order to identify a plurality of compatible profiles from the plurality of product profiles (Step D). The product attributes of each compatible profile are shared amongst each other. In more detail, the plurality of compatible profiles is two or more product profiles that include at least one shared product attribute. Similarly, the remote server compares each product profile amongst each other in order to identify a plurality of incompatible profiles from the plurality of product profiles (Step E). The product attributes of each incompatible profile are in conflict amongst each other. In more detail, the plurality of incompatible profiles is two or more product profiles that do not have any shared product attributes. The remote server generates at least one processing instruction for each product profile in accordance to the product attributes for each product profile, if the compatible profiles are identified during Step D, and/or if the incompatible profiles are identified during Step E. In more detail, the processing instruction includes details on how the products associated to each product profile must be cleaned and verified and the production line order of the products associated to each product profile. Finally, the corresponding user PC device displays the processing instruction for each product profile (Step G). In more detail, the corresponding user PC device outputs at least one assessment report and at least one work order for the product profiles which can be used to instruct employees on how to clean and verify products. The assessment report may be printed, verified, and signed off to also be printed to meet regulatory documentation requirements. Furthermore, the work order can include sanitation work, sanitation verifications, line set up, run rules, as well as Personal Protective Equipment (PPE) requirements. In alternate embodiments, the present invention can provide other reports or results from the assessments for the users to utilize when assessing line changeovers.

In order for a user to select attribute information for each product profile, the following subprocess is executed. A plurality of pre-defined attributes is stored on the remote server. The plurality of pre-defined attributes is selected from a group consisting of: a plurality of cleaning levels; a plurality of cleaning verification levels; a plurality of ancillary production equipment requirements; and a plurality of quality control equipment requirements. The corresponding user PC device prompts the user account to select at least one specific pre-defined attribute for at least one specific product profile. The specific pre-defined attribute is from the plurality of pre-defined attributes and the specific product profile is from the plurality of product profiles. This step provides the user with the option to choose from a list of pre-defined attributes for each product associated with a product profile. The pre-defined attribute for the specific product profile is relayed from the corresponding user PC device to the remote server, if the specific pre-defined attribute for the specific product profile is selected by the user account. In more detail, the remote server has received input from the user associated with the user account and can prepare to process the attribute information. The remote server then appends the specific pre-defined attribute into the plurality of product attributes for the specific product profile during Step C. The specific pre-defined attribute for the specific product profile is now included into the plurality of attributes for Steps D and E. Thus, the present invention provides one or more forms accessible to the user to input the necessary product information. The one or more forms further facilitate the ease of understanding by any user of various skill levels. In alternate embodiments, the present invention can utilize other product information, requirement information, or certification information. The present invention can provide additional tools to help the user input the data or understand the filed data.

Alternatively and in order for the user to fill out attribute information for each product profile, the following subprocess is executed. The corresponding user PC device prompts the user account to enter at least one specific user-defined attribute for at least one specific product profile. The specific product profile is from the plurality of product profiles. The user-defined attribute is selected from a group consisting of: a cleaning level; a cleaning verification level; an ancillary production equipment requirement; and a quality control equipment requirement. In more detail, the user is given the option to input attribute information for each product profile. The specific user-defined attribute is relayed from the corresponding user PC device to the remote server, if the specific user-defined attribute for the specific product profile is entered by the user account. In more detail, the remote server has received input from the user associated with the user account and can prepare to process the attribute information. The remote server then appends the specific user-defined attribute into the plurality of product attributes for the specific product profile during Step C. The specific user-defined attribute for the specific product profile is now included into the plurality of attributes for Steps D and E. Thus, the present invention provides one or more forms accessible to the user to input the necessary product information. The one or more forms further facilitate the ease of understanding by any user of various skill levels. In alternate embodiments, the present invention can utilize other product information, requirement information, or certification information. The present invention can provide additional tools to help the user input the data or understand the filed data.

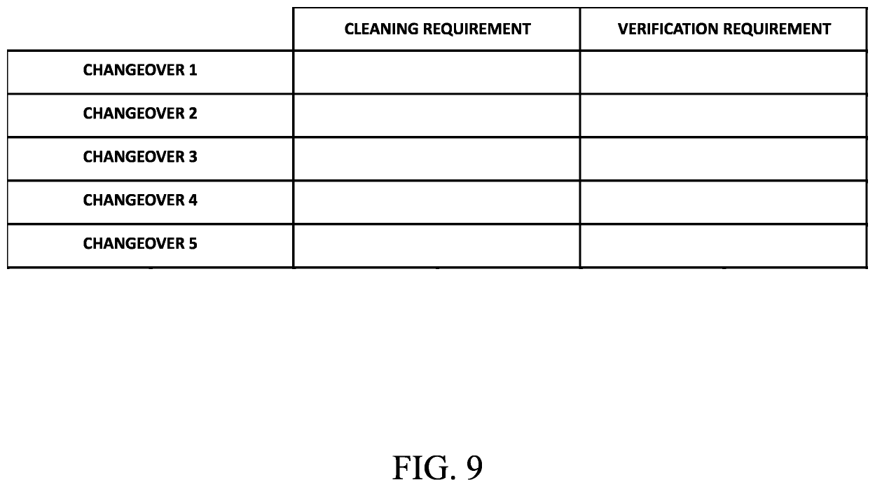

The present invention provides a changeover assessment tool which is executed by the following subprocess. The corresponding user PC device prompts the user account to select a previous product profile and a subsequent product profile after Step F. The previous product profile and the subsequent product profile are from the plurality of product profiles. In more detail, the user is given the option of selecting a first product and a next product in a production line. The previous product profile and the subsequent product profile is relayed from the corresponding user PC device to the remote server, if the previous product profile and the subsequent product profile is selected by the user account. In more detail, the remote server has received input from the user associated with the user account and is prepared to generate the changeover assessment. The remote server then generates at least one changeover assessment between the previous product profile and the subsequent product profile in accordance to the processing instruction for the previous product profile and in accordance to the processing instruction for the subsequent product profile. The changeover assessment provides one or more reports including assessment results detailing food safety considerations, product attribute considerations, and certification considerations. Finally, the corresponding user PC device displays the changeover instruction. With reference to FIG. 9, the changeover assessment can be displayed with one or more tables that include attribute information and compatibility information between the previous product profile and the subsequent product profile.

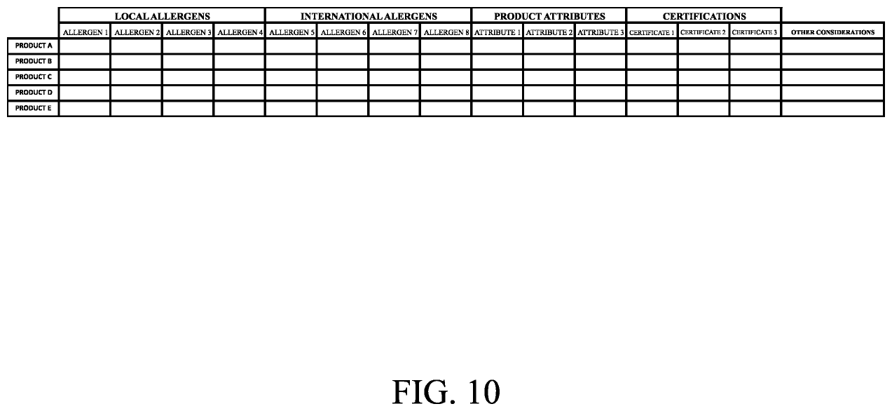

The present invention provides a scheduling planner tool which is executed by the following subprocess. The scheduling planner tool serves as a manufacturing scheduler to plan a group of production runs more efficiently. The corresponding user PC device prompts the user account to select a plurality of specific profiles after Step F. The plurality of specific profiles is from the plurality of product profiles. In more detail, the user is given the option to enter multiple products to simulate a realistic production-line schedule. The plurality of specific profiles is relayed from the corresponding user PC device to the remote server, if the plurality of specific profiles is selected by the user account. In more detail, the remote server has received input from the user associated with the user account and is prepared to generate a simulated production-line schedule. The remote server then generates a simulated production-line schedule for the plurality of specific profiles in accordance to the processing instruction for each specific profile. The simulated production-line schedule reduces the number of changeovers by grouping similar products. By simplifying and automating the changeover process, lesser-skilled users can determine changeover requirements, which in-turn allows for a manufacturing operation to run more efficiently. For example, the simulated production-line schedule indicates similar local or international allergens, individual product attributes, shared certifications, as well as other changeover considerations. Further, the simulated production-line scheduler allows for standardization of changeover planning and reduces potential for error, all of which would contribute to making food safer for consumers. Finally, the corresponding user PC device displays the simulated production-line schedule for the plurality of specific profiles. With reference to FIG. 10, the simulated production-line schedule can be displayed by one or more tables that includes at least one production line order, changeover information, and compatibility information.

Although the invention has been explained in relation to its preferred embodiment, it is to be understood that many other possible modifications and variations can be made without departing from the spirit and scope of the invention as hereinafter claimed.

What is claimed is:

1. A method of managing requirements for foodstuffs with processing line changes, the method comprising the steps of:
(A) providing at least one user account managed by at least one remote server, wherein the user account is associated with a corresponding user personal computing (PC) device;
(B) prompting the user account to enter a plurality of product profiles with the corresponding user PC device, wherein each product profile includes at least one product attribute;
(C) retrieving a plurality of product attributes for each product profile with the remote server, if the product profiles are entered by the user account;
(D) comparing each product attribute for each product profile amongst each other with the remote server in order to identify a plurality of compatible profiles from the plurality of product profiles, wherein the product attributes of each compatible profile are shared amongst each other;
(E) comparing each product attribute for each product profile amongst each other with the remote server in order to identify a plurality of incompatible profiles from the plurality of product profiles, wherein the product attributes of each incompatible profile are in conflict amongst each other;
(F) generating at least one processing instruction for each product profile in accordance to the product attributes for each product profile with the remote server, if the compatible profiles are identified during step (D), and/or if the incompatible profiles are identified during step (E); and
(G) displaying the processing instruction for each product profile with the corresponding user PC device.

2. The method of managing requirements for foodstuffs with processing line changes, the method as claimed in claim 1 comprising the steps:
providing a plurality of pre-defined attributes stored on the remote server;
prompting the user account to select at least one specific pre-defined attribute for at least one specific product profile with the corresponding user PC device, wherein the specific pre-defined attribute is from the plurality of pre-defined attributes, and wherein the specific product profile is from the plurality of product profiles;
relaying the specific pre-defined attribute for the specific product profile from the corresponding user PC device to the remote server, if the specific pre-defined attribute for the specific product profile is selected by the user account; and
appending the specific pre-defined attribute into the plurality of product attributes for the specific product profile with the remote server during step (C).

3. The method of managing requirements for foodstuffs with processing line changes, the method as claimed in claim 2, wherein the plurality of pre-defined attributes is selected from a group consisting of: a plurality of cleaning levels; a plurality of cleaning verification levels; a plurality of ancillary production equipment requirements; and a plurality of quality control equipment requirements.

4. The method of managing requirements for foodstuffs with processing line changes, the method as claimed in claim 1 comprising the steps:
prompting the user account to enter at least one specific user-defined attribute for at least one specific product profile with the corresponding user PC device, wherein the specific product profile is from the plurality of product profiles;
relaying the specific user-defined attribute for the specific product profile from the corresponding user PC device to the remote server, if the specific user-defined attribute for the specific product profile is entered by the user account; and
appending the specific user-defined attribute into the plurality of product attributes for the specific product profile with the remote server during step (C).

5. The method of managing requirements for foodstuffs with processing line changes, the method as claimed in claim 4, wherein the user-defined attribute is selected from a group consisting of: a cleaning level; a cleaning verification level;
an ancillary production equipment requirement; and a quality control equipment requirement.

6. The method of managing requirements for foodstuffs with processing line changes, the method as claimed in claim 1 comprising the steps:
prompting the user account to select a previous product profile and a subsequent product profile with the corresponding user PC device after step (F), wherein the previous product profile and the subsequent product profile are from the plurality of product profiles;
relaying the previous product profile and the subsequent product profile from the corresponding user PC device to the remote server, if the previous product profile and the subsequent product profile is selected by the user account;
generating at least one changeover assessment between the previous product profile and the subsequent product profile in accordance to the processing instruction for the previous product profile and in accordance to the processing instruction for the subsequent product profile with the remote server; and
displaying the changeover assessment with the corresponding user PC device.

7. The method of managing requirements for foodstuffs with processing line changes, the method as claimed in claim 1 comprising the steps:
prompting the user account to select a plurality of specific profiles with the corresponding user PC device after step (F), wherein the plurality of specific profiles is from the plurality of product profiles;
relaying the plurality of specific profiles from the corresponding user PC device to the remote server, if the plurality of specific profiles is selected by the user account;
generating a simulated production-line schedule for the plurality of specific profiles in accordance to the processing instruction for each specific profile with the remote server; and
displaying the simulated production-line schedule for the plurality of specific profiles with the corresponding user PC device.
